(19) United States
(12) Patent Application Publication
(10) Pub. No.: US 2024/0021309 A1
(43) Pub. Date: Jan. 18, 2024

(54) Method for Providing Information about Schizophrenia, and Device for Providing Information about Schizophrenia by Using Same

(71) Applicant: B-Wave Co., Ltd., Goyang-si (KR)

(72) Inventors: Seung Hwan Lee, Goyang-si (KR); Jeong Youn Kim, Seoul (KR)

(73) Assignee: B-Wave Co., Ltd., Goyang-si, GY (KR)

(21) Appl. No.: 18/283,878

(22) PCT Filed: Mar. 23, 2022
(86) PCT No.: PCT/KR2022/004034

§ 371 Date: Sep. 25, 2023

(30) Foreign Application Priority Data
Mar. 25, 2021 (KR) 10-2021-0039131

(51) Int. Cl. G16H 50/20; A61B 5/369; A61B 5/00

(52) U.S. Cl. CPC G16H 50/20; A61B 5/369; A61B 5/7225; A61B 5/7275; A61B 5/7264

(57) ABSTRACT

The present invention provides a method for providing information about schizophrenia, implemented by a processor, the method comprising: receiving an individual's brain wave data; generating brain activity data based on the brain wave data; determining whether the individual's schizophrenia has occurred, using a first classification model configured to classify schizophrenia based on the brain activity data; and determining subtypes of schizohrenia using a second classification model, and a device using the method.

### RELATED APPLICATIONS, INCORPORATIONS BY REFERENCE, AND CLAIMS OF PRIORITY

This application claims the benefit of priority to PCT Application No. PCT/KR2022/004034 entitled “METHOD FOR PROVIDING INFORMATION ABOUT SCHIZOPHRENIA, AND DEVICE FOR PROVIDING INFORMATION ABOUT SCHIZOPHRENIA BY USING SAME,” filed on Mar. 23, 2022, which claims priority to Korean National Application No. 10-2021-0039131 entitled “METHOD FOR PROVIDING INFORMATION ABOUT SCHIZOPHRENIA, AND DEVICE FOR PROVIDING INFORMATION ABOUT SCHIZOPHRENIA BY USING SAME,” filed on Mar. 25, 2021 (now issued as Korean Patent No. 10-2502399-0000 on Feb. 23, 2023). All the aforementioned applications and patents are hereby incorporated by reference in their entirety.

### TECHNICAL FIELD

The present invention relates to a method for providing information about schizophrenia and a device for providing information about schizophrenia using the same.

### BACKGROUND ART

Schizophrenia, one of mental disorders, is also called a mental illness and is a metal disease that causes a wide range of clinical abnormalities over various aspects of personality, including thinking, emotions, perception, and behavior.

Meanwhile, although many clear organic and functional abnormalities of schizophrenia have been known, schizophrenia is not diagnosed on a pathological basis and has been diagnosed based on superficial symptoms.

To solve these limitations, many neurophysiological studies are being conducted on causes and mechanisms of schizophrenia.

In particular, it can be important to develop optimal classification criteria of schizophrenia for accurate diagnosis of schizophrenia.

Therefore, there is a continuous need for the development of new diagnostic criteria for schizophrenia, capable of improving accuracy of diagnosis, and systems for providing information thereon.

The description of the related art has been prepared to facilitate understanding of the disclosure. It should not be construed as acknowledging that matters described in the description of the related art exist as prior arts.

### DETAILED DESCRIPTION OF THE INVENTION

### Technical Problem

Meanwhile, for diagnosis of schizophrenia, functional magnetic resonance imaging (fMRI), which shows unique characteristics of each disorder, has emerged.

Meanwhile, fMRI may cause patients to complain of anxiety or fear during a diagnostic process. Further, fMRI still has many limitations when applied to the diagnosis of schizophrenia, such as high analysis costs and spatial and temporal limitations.

Meanwhile, the inventors of the present invention have paid attention to the fact that changes in biological signals will precede as part of human body's responses, in relation to schizophrenia.

In particular, the inventors of the present invention have paid attention to changes in brain wave data in relation to occurrence of schizophrenia, and were able to recognized that the use of brain wave data can overcome limitations of fMRI analysis described above.

More specifically, the inventors of the present invention were able to recognize that it is possible to extract features related to schizophrenia from brain wave signals and by using the features, schizophrenia can be classified with higher reliability.

As a result, the inventors of the present invention were able to develop a system for providing information regarding schizophrenia based on the brain wave signals.

Meanwhile, the inventors of the present invention were able to recognize that the application of not only brain wave data which can be obtained from a sensor for brain wave signals, but also brain activity data of source activity that is activated together, can contribute to accurate diagnosis of schizophrenia.

In particular, the inventors of the present invention were able to apply the brain activity data to the system for providing information, in consideration of that the brain activity data can reflect functional neurological measure values.

Furthermore, the inventors of the present invention were able to apply a classification model learned from brain activity data to predict schizophrenia to the system for providing information in order to provide highly reliable information.

As a result, the inventors of the present invention were able to confirm that the occurrence of schizophrenia can be classified with high accuracy by applying the classification model.

In particular, the inventors of the present invention were able to confirm that detailed classification of schizophrenia is possible with respect to an additional classification model configured to determine subtypes according to levels of schizophrenia symptoms.

Therefore, problems to be solved by the present invention are to provide a method and a device for providing information about schizophrenia, configured to determine whether an individual's schizophrenia has occurred, by using brain wave data obtained from the individual, brain activity data, and further a classification model.

The problems of the present invention are not limited to the problems mentioned above, and other problems not mentioned will be clearly understood by those skilled in the art from the description below.

### Technical Solution

In order to solve the problems described above, a method for providing information about schizophrenia according to an exemplary embodiment of the present invention is provided. The method for providing information according to an exemplary embodiment of the present invention includes receiving an individual's brain wave data; generating brain activity data based on the brain wave data; determining whether the individual's schizophrenia has occurred, using a first classification model configured to classify schizophrenia by taking the brain activity data as input; and determining symptoms of the individual's schizohrenia using a second classification model configured to classify symptoms of schizophrenia by taking the brain activity data as input.

According to characteristics of the present disclosure, the second classification model may be at least one of a positive symptom classification model configured to classify levels of positive symptoms by taking the brain activity data as input, a negative symptom classification model configured to classify levels of negative symptoms by taking the brain activity data as input, and a cognitive/disorganization symptom classification model configured to classify levels of cognitive/disorganization symptoms by taking the brain activity data as input.

According to another characteristic of the present disclosure, the method for providing information may further include, after the determining of the symptoms of schizophrenia, determining a prognosis of the individual according to the symptoms of the individual's schizophrenia.

According to still another characteristic of the present disclosure, the method for providing information may further include, after the generating of the brain activity data, extracting features of the brain activity data, wherein the determining of whether schizophrenia has occurred may include determining whether the individual' schizophrenia has occurred based on the features, using the first classification model.

According to still another characteristic of the present disclosure, the brain activity data includes a plurality of pieces of brain activity data, and wherein the extracting of the features may include determining functional connectivity between the plurality of pieces of brain activity data, and determining the features between the pieces of brain activity data based on network structural features of the functional connectivity.

According to still another characteristic of the present disclosure, the determining of the functional connectivity may include determining connectivity of a phase locking value (PLV) for each of the plurality of pieces of brain activity data, and the determining of the features between the pieces of brain activity data may include determining the features based on a clustering coefficient and a path length for the connectivity of the PLV for each brain activity data.

According to still another characteristic of the present disclosure, the method for providing information may further include filtering the brain activity data based on a band pass filter, which is performed after the generating of the brain activity data.

According to still another characteristic of the present disclosure, the brain wave data may be defined as brain wave data obtained in a resting state.

According to still another characteristic of the present disclosure, the generating of the brain activity data may include converting the brain wave data into the brain activity data, by using at least one of MNE (minimum-norm estimate), LORETA (low-resolution brain electromagnetic tomography), sLORETA (Standardized low-resolution brain electromagnetic tomography), eLORETA (Exact resolution brain electromagnetic tomography), and dSPM (Dynamic statistical parametric mapping).

According to still another characteristic of the present disclosure, the brain activity data may include current source density (CSD) in at least one brain region among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal. may be, preferably, CDS or source activity in frontal lobe, insula, temporal lobe, occipital lobe, tempo-occipital lobe, parietal lobe, and limbic lobe.

In order to solve the problems described above, a device for providing information about schizophrenia according to an exemplary embodiment of the present invention is provided. The device for providing information according to an exemplary embodiment of the present invention includes a communication unit configured to receive an individual's brain wave data; and a processor connected to communicate with the communication unit. In this case, the processor is configured to, based on the brain wave data, generate brain activity data, determine whether the individual's schizophrenia has occurred, using a first classification model configured to predict whether schizophrenia has occurred by taking the brain activity data as input, and when schizophrenia is predicted, determine symptoms of the individual's schizohrenia using a second classification model configured to classify symptoms of schizophrenia by taking the brain activity data as input.

According to characteristics of the present disclosure, the second classification model may be at least one of a positive symptom classification model configured to classify levels of positive symptoms by taking the brain activity data as input, a negative symptom classification model configured to classify levels of negative symptoms by taking the brain activity data as input, and a cognitive/disorganization symptom classification model configured to classify levels of cognitive/disorganization symptoms by taking the brain activity data as input.

According to still another characteristic of the present disclosure, the processor may be further configured to determine a prognosis of the individual according to the symptoms of the individual's schizophrenia.

According to still another characteristic of the present disclosure, the processor may be further configured to extract features of the brain activity data, and determine whether the individual' schizophrenia has occurred based on the features, using the first classification model. includes a plurality of pieces of brain activity data, and the processor may be configured to determine functional connectivity between the plurality of brain activity data and determine the features between the pieces of brain activity data based on network structural features of the functional connectivity.

According to still another characteristic of the present disclosure, the processor may be configured to determine connectivity of a phase locking value (PLV) for each of the plurality of pieces of brain activity data, and determine the features based on a clustering coefficient and a path length for the connectivity of the PLV for each brain activity data.

According to still another characteristic of the present disclosure, the processor may be further configured to filter the brain activity data based on a band pass filter.

According to still another characteristic of the present disclosure, the brain wave data may be defined as brain wave data obtained in a resting state.

According to still another characteristic of the present disclosure, the processor may be configured to convert the brain wave data into the brain activity data, by using at least one of MNE (minimum-norm estimate), LORETA (low-resolution brain electromagnetic tomography), sLORETA (Standardized low-resolution brain electromagnetic tomography), eLORETA (Exact resolution brain electromagnetic tomography), and dSPM (Dynamic statistical parametric mapping).

According to still another characteristic of the present disclosure, the brain activity data may include current source density (CSD) in at least one brain region among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal.

According to still another characteristic of the present disclosure, the brain activity data may be CDS or source activity in frontal lobe, insula, temporal lobe, occipital lobe, tempo-occipital lobe, parietal lobe, and limbic lobe.

Specific details of other embodiments are included in the detailed description and drawings of the specification.

### Advantageous Effects

The present invention can contribute to a highly reliable diagnosis of schizophrenia by providing a system that uses brain wave data capable of being obtained from a sensor for brain wave signals, and further brain activity data of source activity.

Accordingly, the present invention can overcome limitations of an analysis method such as fMRI, which still has many limitations, such as providing of low-reliability information, high analysis costs, and spatial and temporal constraints.

Furthermore, the present invention can provide highly reliable information about occurrence of schizophrenia by providing a system for providing information, which uses a first classification model learned from brain activity data to predict schizophrenia and a second classification model learned to classify subtypes according to symptoms of schizophrenia.

Accordingly, medical staff can obtain information about an individual suspected, so that it is possible to perform continuous monitoring of the individual suspected of having schizophrenia and to easily predict a treatment prognosis.

Furthermore, the present invention can contribute to early diagnosis of schizophrenia and a good treatment prognosis thereof by providing information on whether schizophrenia has occurred.

The effects according to the present invention are not limited to the details exemplified above, and further various effects are included within the present invention.

### BRIEF DESCRIPTION OF DRAWINGS

FIG. 1A is a schematic diagram illustrating a system for providing information about schizophrenia using bio-signal data according to an exemplary embodiment of the present invention.

FIG. 1B is a schematic diagram illustrating a device for providing information about schizophrenia according to an exemplary embodiment of the present invention.

FIG. 1C is a schematic diagram illustrating a user mobile device that receives information from the device for providing information about schizophrenia according to an exemplary embodiment of the present invention.

FIG. 2A is a schematic flowchart illustrating a method for determining whether schizophrenia has occurred and subtypes thereof based on brain activity data of an individual in the device for providing information about schizophrenia according to an exemplary embodiment of the present invention.

FIG. 2B exemplarily illustrates a procedure for determining whether schizophrenia has occurred and subtypes thereof in the device for providing information about schizophrenia according to an exemplary embodiment of the present invention.

FIGS. 3A and 3B illustrate information on an individual for evaluation of a first classification model and a second classification model that are applied to the device for providing information about schizophrenia according to an exemplary embodiment of the present invention.

FIGS. 3C to 3F show evaluation results of the first classification model and the second classification model that are applied to the device for providing information about schizophrenia according to an exemplary embodiment of the present invention.

### BEST MODE OF THE INVENTION

Advantages and features of the present invention and methods to achieve them will become apparent from descriptions of embodiments herein below with reference to the accompanying drawings. However, the present invention is not limited to the embodiments disclosed herein but may be implemented in various different forms. The embodiments are provided to make the description of the present invention thorough and to fully convey the scope of the present invention to those skilled in the art. It is to be noted that the scope of the present invention is defined only by the claims. In connection with the description of drawings, the same or like reference numerals may be used for the same or like elements.

In the disclosure, expressions “have,” “may have,” “include” and “comprise,” or “may include” and “may comprise” used herein indicate presence of corresponding features (for example, elements such as numeric values, functions, operations, or components) and do not exclude the presence of additional features.

In the disclosure, expressions “A or B,” “at least one of A or/and B,” or “one or more of A or/and B,” and the like used herein may include any and all combinations of the associated listed items. For example, the “A or B,” “at least one of A and B,” or “at least one of A or B” may refer to all of case (1) where at least one A is included, case (2) where at least one B is included, or case (3) where both of at least one A and at least one B are included.

The expressions, such as “first,” “second,” and the like used herein, may refer to various elements, but do not limit the order and/or priority of the elements. Furthermore, such expressions may be used to distinguish one element from another element but do not limit the elements. For example, a first user device and a second user device indicate different user devices regardless of the order or priority. For example, without departing from the scope of the present invention, a first element may be referred to as a second element, and similarly, a second element may also be referred to as a first element.

It will be understood that when an element (for example, a first element) is referred to as being “(operatively or communicatively) coupled with/to” or “connected to” another element (for example, a second element), it can be understood as being directly coupled with/to or connected to another element or coupled with/to or connected to another element via an intervening element (for example, a third element). On the other hand, when an element (for example, a first element) is referred to as being “directly coupled with/to” or “directly connected to” another element (for example, a second element), it should be understood that there is no intervening element (for example, a third element) therebetween.

According to the situation, the expression “configured to (or set to)” used herein may be interchangeably used with, for example, the expression “suitable for,” “having the capacity to,” “designed to,” “adapted to,” “made to,” or “capable of”. The term “configured to (or set to)” may not necessarily mean only “specifically designed to” in hardware. Instead, the expression “a device configured to” in any situation may mean that the device is “capable of operating together with another device or other components. For example, a “processor configured to (or set to) perform A, B, and C” may mean a dedicated processor (for example, an embedded processor) for performing a corresponding operation or a generic-purpose processor (for example, a central processing unit (CPU) or an application processor) which may perform corresponding operations by executing one or more software programs which are stored in a memory device.

Terms used in the present invention are used to describe specified embodiments of the present invention and are not intended to limit the scope of other embodiments. The terms of a singular form may include plural forms unless otherwise specified. All the terms used herein, which include technical or scientific terms, may have the same meaning that is generally understood by a person skilled in the art. It will be further understood that terms which are defined in a dictionary among terms used in the disclosure, can be interpreted as having the same or similar meanings as those in the relevant related art and should not be interpreted in an idealized or overly formal way, unless expressly defined in the present invention. In some cases, even in the case of terms which are defined in the specification, they cannot be interpreted to exclude embodiments of the present invention.

Features of various exemplary embodiments of the present invention may be partially or fully combined or coupled. As will be clearly appreciated by those skilled in the art, technically various interactions and operations are possible, and respective embodiments may be implemented independently of each other or may be implemented together in an associated relationship.

For clarity of interpretation of the present specification, terms used herein will be defined below.

As used herein, the term “schizophrenia” may refer to a mental disease that causes a wide range of clinical abnormalities over various aspects of personality, including thinking, emotions, perception, and behavior.

As used herein, the term “brain wave data” may mean an EEG (electroencephalogram) signal value recorded in a sensor that detects brain waves.

Meanwhile, since the brain wave data may be a signal or signal value obtained from a sensor, it may be interpreted in the same sense as sensor data within the present specification.

According to characteristics of the present invention, the brain wave data may include brain wave data measured from at least one electrode among Fp1, Fp2, F3, Fz, F4, F8, T7, C3, C4, Cz, T8, P7, P3, Pz, P4, P8, O1, O2, FCz, TP9, TP10, Oz, AFz, F7, Fpz, AF7, AF3, AF4, AF8, F9, F5, F1, F2, F6, F10, FT9, FT7, FC5, FC3, FC1, FC2, FC4, FC6, FT8, FT10, C5, C1, C2, C6, TP7, CP5, CP3, CP1, CPz, CP2, CP4, CP6, TP8, P9, P5, P1, P2, P6, P10, PO9, PO7, PO3, POz, PO4, PO8, PO10, O9, Iz, O10, F11, F12, FT11, FT12, TP11, TP12, PO11, PO12, P11, P12, I11, I12, and Hz.

According to characteristics of the present invention, the brain wave data may be brain wave data obtained in a resting state without stimulation that is applied to an individual, but is not limited thereto.

As used herein, the term “brain activity data” may mean data on source activity that is activated while stimulation is output. In this case, the source activity may correspond to current source density (CSD) in a brain active region.

For example, according to another characteristic of the present invention, the brain activity data may include current source density (CSD) or source activity in at least one brain region selected from among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal.

Preferably, the brain activity data may be CDS or source activity in frontal lobe, insula, temporal lobe, occipital lobe, tempo-occipital lobe, parietal lobe, and limbic lobe, but is not limited thereto.

Since the brain activity data can be defined as source activity, it can be interpreted to have the same meaning as source data within the specification of this application.

Meanwhile, the brain activity data may be generated based on the brain wave data aforementioned.

In addition, the brain activity data can be obtained by estimating source activity of voxels corresponding to a source space, using at least one of MNE (minimum-norm estimate), wMNE (weighted MNE), LORETA (low-resolution brain electromagnetic tomography), sLORETA (standardized low-resolution brain electromagnetic tomography), eLORETA (exact resolution brain electromagnetic tomography), dSPM (dynamic statistical parametric mapping), LCMV (linearly constrained minimum variance) beamformers programs-LORETA/sLORETA toolbox, Brainstrom, eConnectome, fieldtrip, and EEGlab.

Preferably, the brain activity data may be data obtained through MNE, but is not limited thereto.

As used herein, the term “features” may refer to parameters according to quantification of a brain neural network.

According to characteristics of the present invention, features can be extracted from the brain activity data.

For example, features of network indices of a clustering coefficient indicating a degree of clustering and a path length indicating a length of a connection path between specific regions can be determined from the brain activity data.

However, the present invention is not limited to thereto, and from the brain activity data, at least one feature can be extracted from among strength of connectivity of a phase locking value (PLV), a degree of network connectivity, nodal and global efficiency for quantifying efficiency of an information processing process within networks, betweeness that identifies a hub, which is an information-dense area in the connection path, closeness, eigenvector centrality, and rich club.

In this case, the features may correspond to connection characteristics of and changes in functional networks of a specific brain region.

As used herein, the term “first classification model” refers to a model that is learned to classify whether schizophrenia has occurred by taking an individual's brain wave data and/or brain activity data obtained from a brain wave measurement device and/or brain electromagnetic tomography as input.

According to characteristics of the present invention, the first classification model may be a model that is configured to output schizophrenia or normality by taking features determined from the brain activity data as input.

As used herein, the term “second classification model” may be a model that is learned to classify subtypes according to symptoms of schizophrenia based on the individual's brain wave data and/or brain activity data obtained from the brain wave measurement device and/or brain electromagnetic tomography.

According to characteristics of the present invention, the second classification model may be a model configured to output subtypes according to levels of symptoms of schizophrenia by taking the features determined from the brain activity data as input.

In this case, the subtypes according to the levels of symptoms of schizophrenia may include a positive symptom group in which a specific condition is more prominent compared to a healthy normal group, a negative symptom group in which a specific condition is reduced compared to the healthy normal group, and furthermore, a cognitive/disorganization symptom group. In this case, the subtypes can be distinguished based on a positive and negative syndrome scale (PANSS), but are not limited thereto.

According to another characteristic of the present invention, the second classification model may include a positive symptom classification model configured to classify levels of positive symptoms by taking the brain activity data as input and/or a negative symptom classification model configured to classify levels of negative symptoms by taking the brain activity data as input and/or a cognitive/disorganization symptom classification model configured to classify levels of cognitive/disorganization symptoms by taking the brain activity data as input.

For example, the positive symptom classification model may be configured to output the symptom as a high positive symptom or low positive symptom according to a level (or severity) of an individual's positive symptom by taking feature parameters of the brain activity data as input. The negative symptom classification model may be configured to output the symptom as a high negative symptom or low negative symptom according to a level (or severity) of the individual's negative symptom according to a level (or severity) of an individual's negative symptom by taking feature parameters of the brain activity data as input. The cognitive/disorganization symptom classification model may be configured to output the symptom as a high cognitive/disorganization symptom or low cognitive/disorganization symptom according to a level (or severity) of the individual's cognitive/disorganization symptom by taking the feature parameters of the brain activity data as input.

However, the second classification model is not limited thereto and may be configured to output a symptom level of the individual's schizophrenia as high, medium, or low, or output it as a probability of the corresponding symptom.

Additionally, the second classification model may be a single model, rather than three independent models that are differentiated according to subtypes, and may be learned to classify the levels of positive symptoms, the levels of negative symptoms, and the levels of cognitive/disorganization symptoms.

According to another characteristic of the present invention, the first classification model and the second classification model may be classification models based on at least one algorithm among linear discriminant analysis (LDA), support vector machine (SVM), decision tree, random forest, AdaBoost (Adaptive Boosting), PLR (Penalized Logistic Regression), GBM (gradient boosting machine), KNN (K-nearest neighborhood), Naive Bayes, and Riemannian classifier.

Preferably, the first classification model and the second classification model may be LDA-based models, but are not limited thereto, and may be based on more diverse learning algorithms.

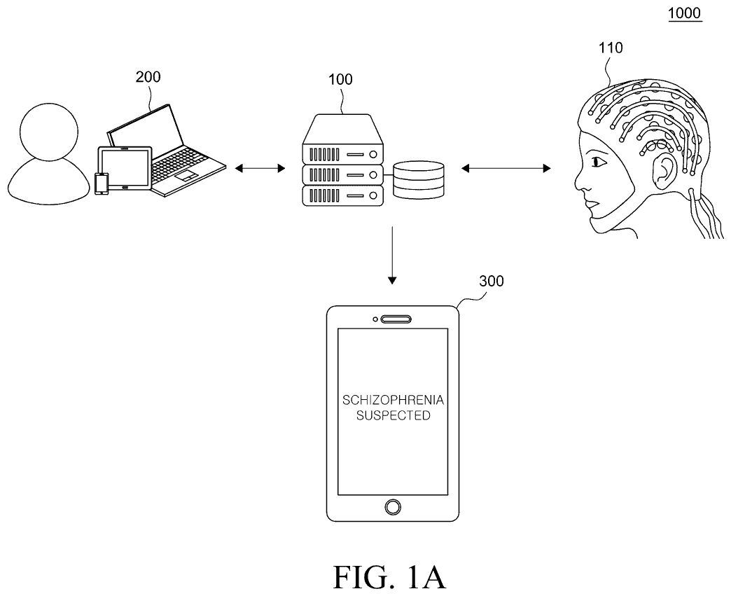

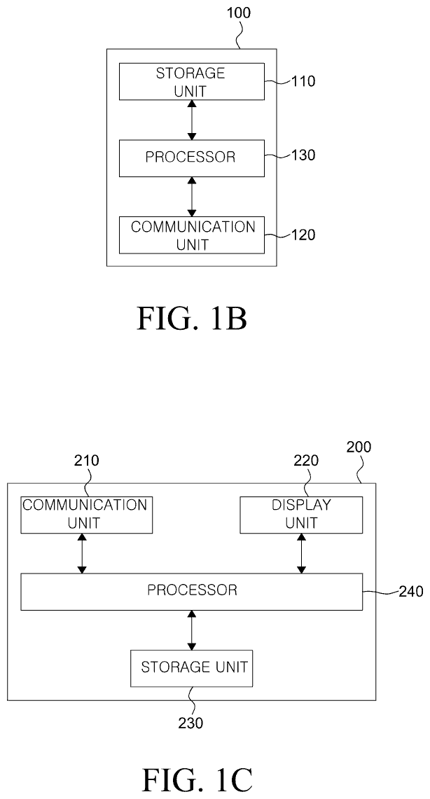

Hereinafter, with reference to FIGS. 1A to 1C, a device for providing information about schizophrenia according to various embodiments of the present invention will be described in detail.

FIG. 1A is a schematic diagram illustrating a system for providing information about schizophrenia using bio-signal data according to an exemplary embodiment of the present invention.

First, referring to FIG. 1A, a system 1000 for providing information may be a system configured to provide information about schizophrenia based on a user's brain waves. In this case, the system 1000 for providing information about schizophrenia may be configured to include a device 100 for providing information about schizophrenia, which is configured to determine whether an individual's schizophrenia has occurred based on brain wave data and/or brain activity data, a user mobile device 200, a medical staff device 300, and a device 400 for measuring brain waves, which is configured to measure brain waves by being in close contact with the user's scalp.

First, the device 100 for providing information about schizophrenia may include a general-purpose computer, a laptop, and/or a data server and the like, that perform various calculations to evaluate whether schizophrenia has occurred based on the user's brain waves provided from the device 400 for measuring brain waves. In this case, the user mobile device 200 may be a device for accessing a web server providing a web page about schizophrenia or a mobile web server providing a mobile website, but is limited thereto. Furthermore, the device 400 for measuring brain waves may be formed of a plurality of electrodes that are configured to externally surround the user's head. Meanwhile, the plurality of electrodes may include at least one standard electrode among Fp1, Fp2, F3, Fz, F4, F8, T7, C3, C4, Cz, T8, P7, P3, Pz, P4, P8, O1, O2, FCz, TP9, TP10, Oz, AFz, F7, Fpz, AF7, AF3, AF4, AF8, F9, F5, F1, F2, F6, F10, FT9, FT7, FC5, FC3, FC1, FC2, FC4, FC6, FT8, FT10, C5, C1, C2, C6, TP7, CP5, CP3, CP1, CPz, CP2, CP4, CP6, TP8, P9, P5, P1, P2, P6, P10, PO9, PO7, PO3, POz, PO4, PO8, PO10, O9, Iz, O10, F11, F12, FT11, FT12, TP11, TP12, PO11, PO12, P11, P12, I11, I12, and Hz.

Specifically, the device 100 for providing information about schizophrenia may be configured to receive brain wave data from the device 400 for measuring brain waves, convert the received brain wave data into brain activity data and extract features therefrom, and then, classify it as schizophrenia or normality, or classify subtypes according to levels of symptoms of schizophrenia.

Optionally, the device 100 for providing information about schizophrenia may be configured to receive the brain activity data from brain electromagnetic tomography (not shown), and classify it as schizophrenia or normality therefrom, or classify subtypes according to levels of symptoms of schizophrenia.

The device 100 for providing information about schizophrenia may provide data that analyzes whether an individual' schizophrenia has occurred and/or a subtype according to a symptom of schizophrenia to the user mobile device 200 and further, to the medical staff device 300.

In this manner, the data provided from the device 100 for providing information about schizophrenia may be provided as a web page through a web browser installed on the user mobile device 200 and/or the medical staff device 300, or may be provided in the form of an application or program. In various embodiments, such data may be provided in the form included in a platform in a client-server environment.

Next, the user mobile device 200 is an electronic device that requests information about occurrence of schizophrenia for an individual and provides a user interface for displaying analysis result data, and may include at least one among a smartphone, a tablet PC (personal computer), or a laptop and/or a PC.

The user mobile device 200 may receive analysis results regarding the occurrence of schizophrenia for the individual from the device 100 for providing information about schizophrenia, and display the received results through a display unit of the user mobile device 200. Here, the analysis results may include a risk of developing schizophrenia as being high, medium, or low, a probability of developing schizophrenia, and the like.

Next, with reference to FIG. 1B, components of the device 100 for providing information about schizophrenia of the present invention will be described in detail.

FIG. 1B is a schematic diagram illustrating a device for providing information about schizophrenia according to an exemplary embodiment of the present invention.

Referring to FIG. 1B, the device 100 for providing information about schizophrenia includes a storage unit 110, a communication unit 120, and a processor 130.

First, the storage unit 110 may store various pieces of data for evaluating whether an individual's schizophrenia has occurred. In various embodiments, the storage unit 110 may include at least one type of storage medium among a flash memory type, a hard disk type, a multimedia card micro type, a card type memory (e.g., an SD or XD memory, etc.), a random-access memory (RAM), a static random-access memory (SRAM), a read-only memory (ROM), an electrically erasable programmable read-only memory (EEPROM), a programmable read-only memory (PROM), a magnetic memory, a magnetic disk, and an optical disk.

The communication unit 120 connects the device 100 for providing information about schizophrenia with an external device so that they can communicate with each other. The communication unit 120 may be connected to the user mobile device 200, the medical staff device 300, and furthermore, the device 400 for measuring brain waves using wired/wireless communications and transmit and receive various pieces of data. Specifically, the communication unit 120 may receive an individual's brain wave data from the device 400 for measuring brain waves and receive brain activity data from brain electromagnetic tomography (not shown). Also, the communication unit 120 may transmit analysis results to the user mobile device 200 and/or the medical staff device 300.

The processor 130 is operatively connected to the storage unit 110 and the communication unit 120, and may perform various commands for analyzing the brain wave data and/or brain activity data for the individual.

Specifically, the processor 130 may receive the brain wave data of the individual from the device 400 for measuring brain waves through the communication unit 120, generate brain activity data based on the received brain wave data, extract features therefrom and evaluate whether the individual's schizophrenia has occurred and further, subtypes according to levels of the symptoms of schizophrenia.

Meanwhile, the processor 130 may be configured to convert the brain wave data into the brain activity data, using at least one among low-resolution brain electromagnetic tomography (LORETA), standardized low-resolution brain electromagnetic tomography (sLORETA), exact resolution brain electromagnetic tomography (eLORETA), minimum-norm estimate (MNE), weighted MNE (wMNE), dynamic statistical parametric mapping (dSPM), linearly constrained minimum variance (LCMV) beamformers programs-LORETA/sLORETA toolbox, brainstrom, eConnectome, fieldtrip and EEGlab.

Moreover, the processor 130 may be based on the first classification model that is configured to classify whether schizophrenia has occurred based on the brain activity data, and further, the second classification model that is configured to classify subtypes of schizophrenia based on the brain activity data.

Accordingly, the user can easily obtain information about his or her mental health through the user mobile device 200 without temporal or spatial constraints. Furthermore, medical staff can obtain information about the individual from the medical staff device 300, so that it is possible to perform continuous monitoring of the individual suspected of having schizophrenia.

In this manner, in the present invention, whether schizophrenia has occurred can be classified with high accuracy and information thereon can be provided, thereby contributing to early diagnosis of schizophrenia and a good treatment prognosis.

Meanwhile, referring to FIG. 1C together, the user mobile device 200 includes a communication unit 210, a display unit 220, a storage unit 230, and a processor 240.

The communication unit 210 connects the user mobile device 200 with an external device so that they communicate with each other. The communication unit 210 is connected to the device 100 for providing information about schizophrenia using wired/wireless communications and can transmit and receive various pieces of data. Specifically, the communication unit 210 may receive an analysis result related to diagnosis of schizophrenia of an individual from the device 100 for providing information about schizophrenia.

The display unit 220 can display various interface screens to display analysis results related to the diagnosis of schizophrenia of the individual.

In various embodiments, the display unit 220 may include a touch screen and may receive, for example, a touch, gesture, proximity, drag, swipe, or hovering input or the like using an electronic pen or a body portion of the user.

The storage unit 230 may store various pieces of data used to provide a user interface for displaying result data. In various embodiments, the storage unit 230 may include at least one type of storage medium among a flash memory type, a hard disk type, a multimedia card micro type, a card type memory (e.g., an SD or XD memory, etc.), a random-access memory (RAM), a static random-access memory (SRAM), a read-only memory (ROM), an electrically erasable programmable read-only memory (EEPROM), a programmable read-only memory (PROM), a magnetic memory, a magnetic disk, and an optical disk.

The processor 240 is operatively connected to the communication unit 210, the display unit 220, and the storage unit 230, and may perform various commands for providing a user interface for displaying result data.

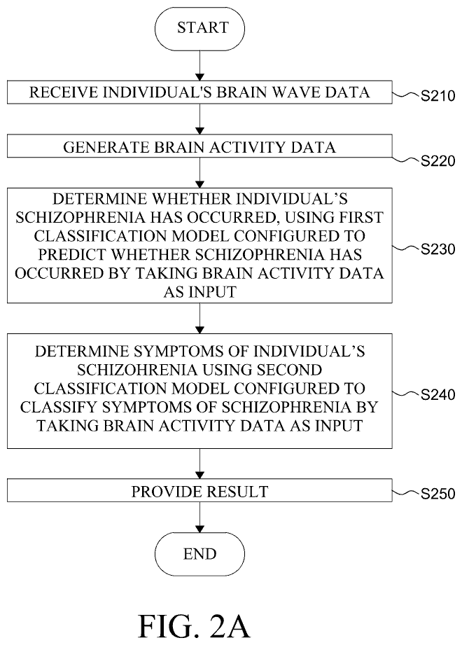

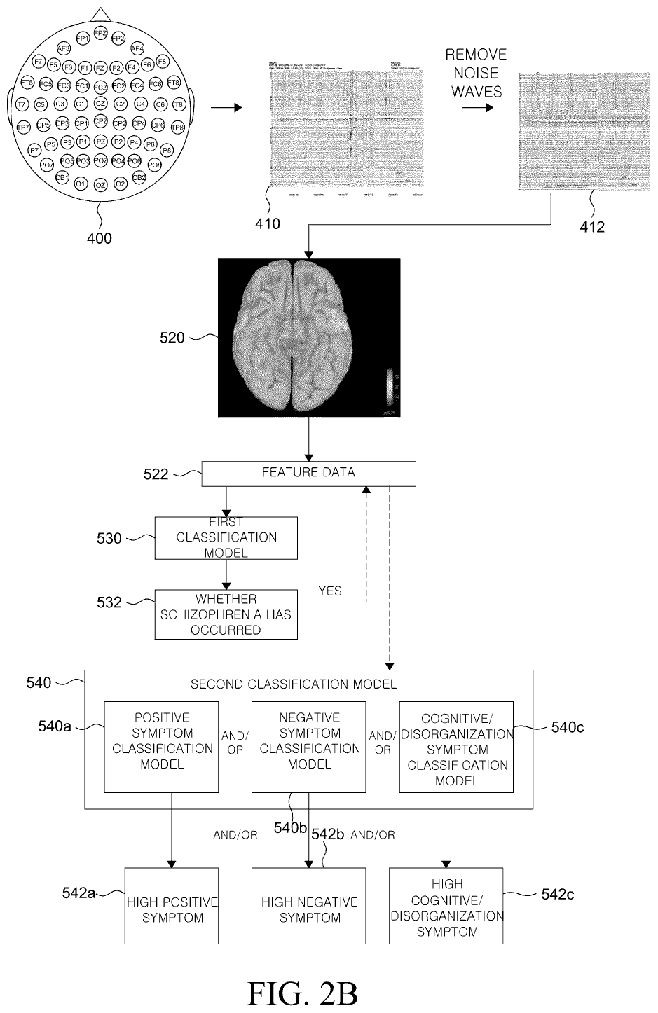

Hereinafter, a method of providing information according to various embodiments of the present invention will be described with reference to FIGS. 2A and 2B.

FIG. 2A is a schematic flowchart illustrating a method for determining whether schizophrenia has occurred and subtypes thereof based on brain activity data of an individual in the device for providing information about schizophrenia according to an exemplary embodiment of the present invention. FIG. 2B exemplarily illustrates a procedure for determining whether schizophrenia has occurred and subtypes thereof in the device for providing information about schizophrenia according to an exemplary embodiment of the present invention.

First, referring to FIG. 2A, an individual's brain wave data is received according to a method for providing information about schizophrenia according to an exemplary embodiment of the present invention in step S210. Next, the brain activity data is generated based on the brain wave data in step S220. Next, whether the individual's schizophrenia has occurred is determined based on the first classification model in step S230. Next, subtypes according to levels of symptoms of schizophrenia are determined by the second classification model in step S240. Finally, a final result is provided in step S250.

More specifically, in the step S210 in which the individual's brain wave data is received, brain wave data obtained in a resting state may be obtained.

For example, in the step S210 in which the individual's brain wave data is received, brain wave data obtained in a resting state measured from at least one electrode among Fp1, Fp2, F3, Fz, F4, F8, T7, C3, C4, Cz, T8, P7, P3, Pz, P4, P8, O1, O2, FCz, TP9, TP10, Oz, AFz, F7, Fpz, AF7, AF3, AF4, AF8, F9, F5, F1, F2, F6, F10, FT9, FT7, FC5, FC3, FC1, FC2, FC4, FC6, FT8, FT10, C5, C1, C2, C6, TP7, CP5, CP3, CP1, CPz, CP2, CP4, CP6, TP8, P9, P5, P1, P2, P6, P10, PO9, PO7, PO3, POz, PO4, PO8, PO10, O9, Iz, O10, F11, F12, FT11, FT12, TP11, TP12, PO11, PO12, P11, P12, I11, I12, and Hz can be obtained.

Meanwhile, if the brain wave data obtained in the step S210 of receiving the individual's brain wave data includes noise waves, the noise waves may be removed.

For example, referring to FIG. 2B together, noise waves from brain wave data 410 received from the device 400 for measuring brain waves may be removed.

Referring to FIG. 2A, again, the brain activity data is generated based on the obtained brain wave data in step S220.

According to characteristics of the present invention, in the step S220 in which the brain activity data is generated, the brain wave data can be converted into the brain activity data by at least one of MNE (minimum-norm estimate), wMNE (weighted MNE), LORETA (low-resolution brain electromagnetic tomography), sLORETA (standardized low-resolution brain electromagnetic tomography), eLORETA (exact resolution brain electromagnetic tomography), dSPM (dynamic statistical parametric mapping), LCMV (linearly constrained minimum variance) beamformers programs-LORETA/sLORETA toolbox, Brainstrom, eConnectome, fieldtrip, and EEGlab. Preferably, in the step S220 in which the brain activity data is generated, the brain activity data is generated by the MNE.

According to another characteristic of the present invention, the brain activity data generated in the step S220 in which the brain activity data is generated may be CDS or source activity in frontal lobe, insula, temporal lobe, occipital lobe, tempo-occipital lobe, parietal lobe, and limbic lobe, but is not limited thereto.

According to another characteristic of the present invention, after the step S220 in which the brain activity data is generated, filtering of the generated brain activity data may be performed. That is, brain activity data for a specific frequency can be obtained through filtering.

For example, in the step S220 in which the brain activity data is generated, the brain activity data is generated by the MNE, and thereafter, brain activity data in a specific frequency domain, more specifically, at delta waves from 1 to 4 Hz, (δ), theta waves (θ) from 4 to 8 Hz, alpha waves (α) from 8 to 12 Hz, alpha 1 waves from 8 to 10 Hz, alpha 2 waves from 10 to 12 Hz, beta waves (β) from 12 to 30 Hz waves, beta 1 waves from 12 to 18 Hz, beta 2 waves from 18 to 22 Hz, beta 3 waves from 22 to 30 Hz, beta 4 waves from 18 to 30 Hz, and gamma waves (γ) from 30 to 55 Hz can be obtained by a band pass filter.

According to another characteristic of the present invention, features may be extracted from the brain activity data after the step S220 in which the brain activity data is generated. At the step, network structural features for the brain activity data can be determined.

According to another characteristic of the present invention, in the step where the features are extracted, functional connectivity between a plurality of pieces of the brain activity data is determined, and features of the brain activity data are determined based on network structural features of the functional connectivity.

According to another characteristic of the present invention, in the step where the features are extracted, connectivity of a PLV (phase locking value) for each of the plurality of pieces of brain activity data is determined, and based on a clustering coefficient and a pass length for the connectivity of the PLV for each of the plurality of pieces of brain activity data, features can be determined.

For example, in the step where the features are extracted, with respect to the filtered brain activity data for a specific frequency, the PLV (phase locking value), which is a synchrony value for examining a change induced in a work in long-distance synchronization of neural activity can be calculated. As a result, a functional connectivity matrix is formed, and the connectivity of the PLV of each of frequency domains (δ, θ, α, β, and γ) is determined. Next, in the step S230 in which the features are extracted from the brain activity data, the path length of the connectivity of the PLV is determined, and/or a clustering coefficient corresponding to a cluster tendency for the connectivity of the PLV is calculated.

That is, as a result of the step in which the features are extracted, network indices that are a plurality of features may be determined from the brain activity data.

Referring to FIG. 2A again, in the step S230 in which whether the individual's schizophrenia has occurred is determined, whether the individual's schizophrenia has occurred is determined based on the brain activity data and further, features extracted from the brain activity data.

According to characteristics of the present invention, in the step S230 in which whether the individual's schizophrenia has occurred is determined, the first classification model may output whether the individual's schizophrenia has occurred by taking feature data extracted from brain activity data as input. Next, depending on whether schizophrenia has occurred, the step S240 in which a subtype of schizophrenia is determined is performed, and the subtype of schizophrenia can be determined by the second classification model.

For example, referring also to FIG. 2B, once feature data 522 (e.g., a path length and/or clustering coefficient) corresponding to a network index for brain activity data 520 is generated, these parameters are input to a first classification model 530 in the step S230 in which whether the individual's schizophrenia has occurred is determined. Next, a classification result 532 is output, and when it is determined that schizophrenia has occurred, feature data 522 is input to a second classification model 540 in the step S240 in which the subtype of schizophrenia is determined.

In this case, the second classification model 540 may be configured to include a positive symptom classification model 540a configured to classify levels of positive symptoms by taking the feature data 522 as input and/or a negative symptom classification model 540b configured to classify levels of negative symptoms by taking the feature data 522 as input and/or a cognitive/disorganization symptom classification model 540c configured to classify levels of cognitive/disorganization symptoms by taking the feature data 522 as input.

That is, in the step S240 in which the subtype of schizophrenia is determined, by the second classification model 540, the level of the positive symptom 542a and/or the level of the negative symptom 542b and/or the level of the cognitive/disorganization symptom 542c are output as ‘high’ or ‘low’, so that the subtype can be determined according to the level of symptoms of the individual.

In this case, the second classification model 540 may be configured to output a symptom level of an individual's schizophrenia as ‘high’, ‘medium’, or ‘low’, output it as ‘high’ or ‘low’, or output it as a probability of the corresponding symptom.

Referring again to FIG. 2A, as a result of the step S230 in which whether the individual's schizophrenia has occurred is determined and the step S240 in which the subtype of schizophrenia is determined, information related to the individual's schizophrenia may be determined. Finally, in the step S250 in which the result is provided, information related to occurrence of schizophrenia, which is determined by the first classification model and the second classification model may be transmitted to the user's mobile device, and further to the medical staff device.

Meanwhile, according to another characteristic of the present invention, treatment prognosis is predicted according to the symptom level of schizophrenia according to the step S240 in which the subtype of schizophrenia is determined, and in the step S250 in which the predicted prognosis is provided, the predicted prognosis can be provided to users or medical staff

For example, referring to FIG. 2B, in the case of an individual whose the level of the positive symptom 542a and/or the level of the negative symptom 542b and/or the level of the cognitive/disorganization symptom 542c are all determined to be ‘high’ by each second classification model 540, it can be predicted that the individual's treatment prognosis will not be good compared to treatment prognosis of an individual whose the level of the positive symptom 542a and/or the level of the negative symptom 542b and/or the level of the cognitive/disorganization symptom 542c are determined to be ‘low’ by the second classification model 540.

Furthermore, it may be possible to monitor a degree of recovery of an individual according to the classification result of the subtype by the second classification model 540 according to a progress of schizophrenia treatment. For example, when it is determined that the individual's schizophrenia has occurred, steps of receiving brain wave data, generating brain activity data, and re-determining whether the individual's schizophrenia has occurred may be repeatedly performed according to the progress of treatment.

Through the method for providing information about schizophrenia according to various embodiments of the present invention, medical staff can obtain information about an individual and thus, can perform continuous monitoring, such as evaluating treatment prognosis for an individual suspected of having schizophrenia.

### Evaluation: Feature Extraction for Schizophrenia Classification and Classification

Performance Evaluation of Device for Providing Information about Schizophrenia

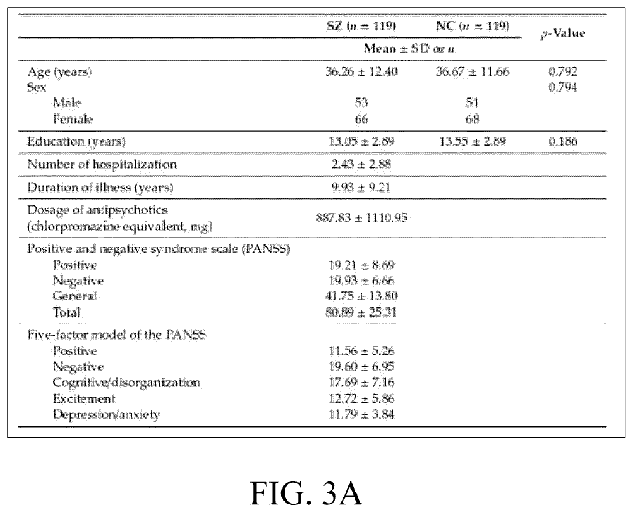

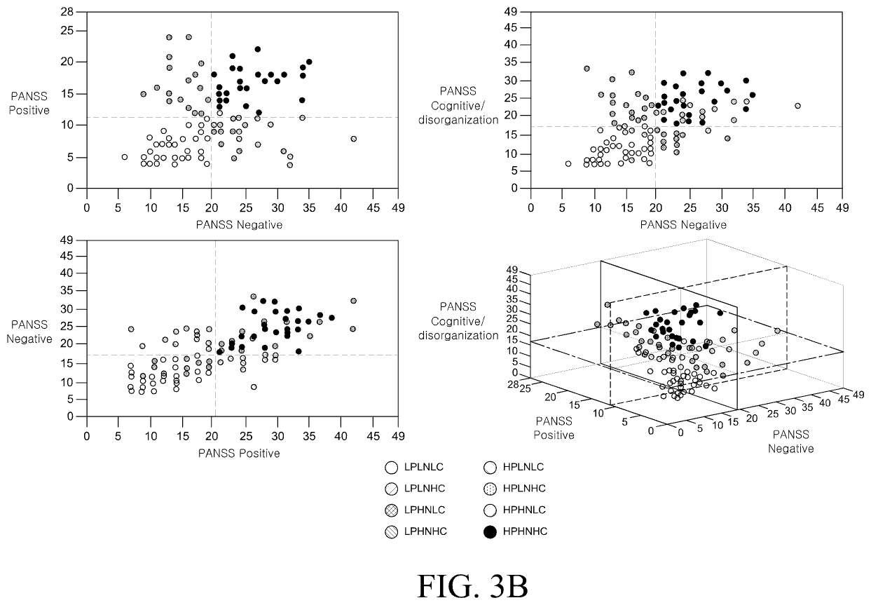

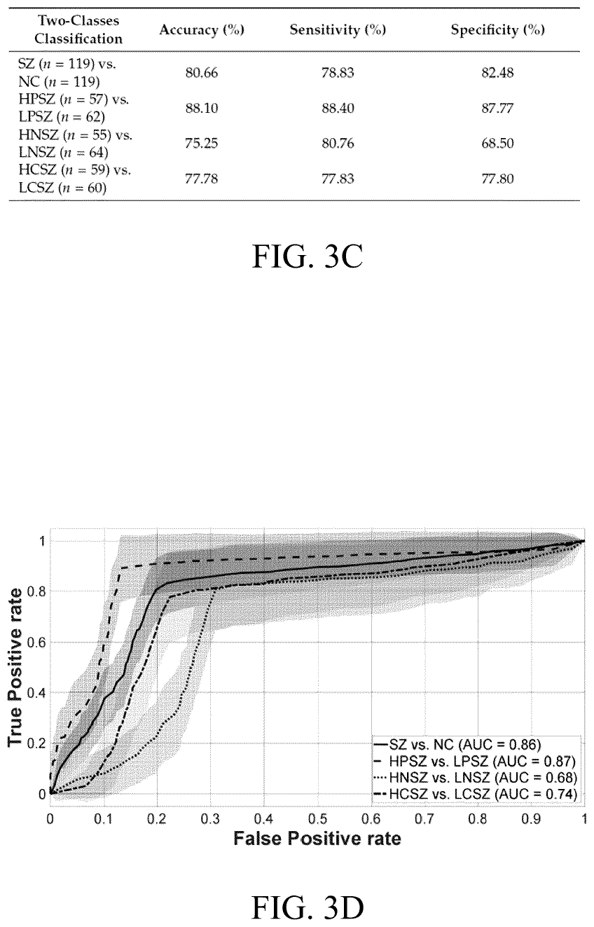

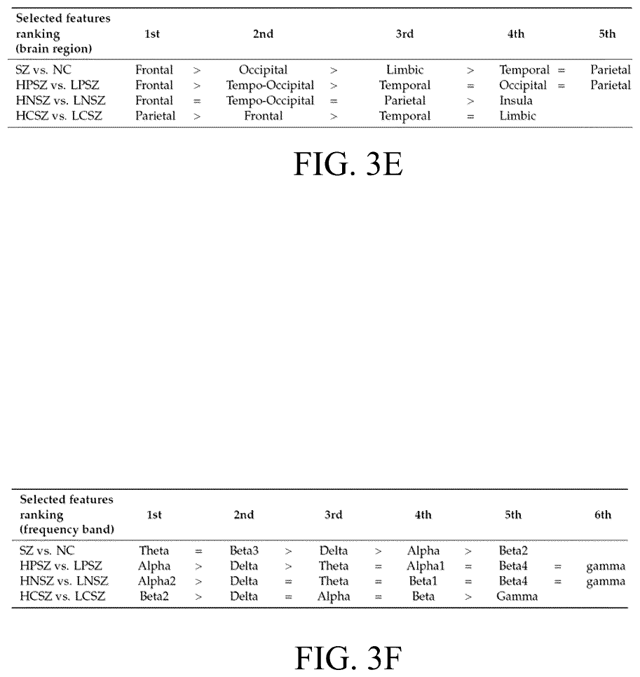

Hereinafter, with reference to FIGS. 3A to 3F, evaluation results of the device for providing information about schizophrenia according to an exemplary embodiment of the present invention will be described.

Referring to FIG. 3A, in this evaluation, brain wave data of a total of 199 individuals with schizophrenia (SZ) and 199 individuals in a normal control (NC) group was used.

In this case, for individuals with schizophrenia, it is shown that an average positive symptom score is 19.21, an average negative symptom score is 19.93, and an average general score is 41.75, according to a positive and negative syndrome scale (PANSS).

In addition, according to PANSS based on five factors, it is shown that an average positive symptom score is 11.56, an average negative symptom score is 19.60, an average cognitive/disorganization symptom score is 17.69, an average excitement symptom score is 12.72, and an average depression/anxiety symptom score is 11.79.

Here, positive symptoms can be evaluated based on P1, P3, P5 and P6 in PANSS, negative symptoms can be evaluated based on N1, N2, N3, N4, N6, G7 and G16 in PANSS, and cognitive/disorganization symptoms can be evaluated based on P2, N5, G9, G10, G11, G13 and G15 in PANSS. Additionally, excitement symptoms can be evaluated based on P4, P7, G8, G12 and G14 in PANSS, and depression/anxiety symptoms can be evaluated based on G2, G3, G4 and G6 in PANSS.

Referring to FIG. 3B, distribution of PANSS scores by subtype for individuals with schizophrenia is shown.

More specifically, individuals with schizophrenia may be distinguished into LPLNLC (low positive, low negative, low cognitive/disorganization), LPLNHC (low positive, low negative, high cognitive/disorganization), LPHNLC (low positive, high negative, low cognitive/disorganization), LPHNHC (low positive, high negative, high cognitive/disorganization), HPLNLC (high positive, low negative, low cognitive/disorganization), HPLNHC (high positive, low negative, high cognitive/disorganization), HPHNLC (high positive, high negative, low cognitive/disorganization), and HPHNHC (high positive, high negative, high cognitive/disorganization).

In this case, a high positive symptom group and a low positive symptom group can be distinguished based on a median in PANSS positivity level of 11 scores, and a high negative symptom group and a low negative symptom group can be distinguished based on a median in PANSS negativity level of 19 scores. Furthermore, a high cognitive/disorganization symptom group and a low cognitive/disorganization symptom group can be distinguished based on a median in PANSS cognitive/disorganization level of 17 scores. However, differentiation of the levels of symptoms of schizophrenia is not limited to the above-described scores and can be performed according to more diverse threshold ranges.

The first classification model classified 119 people in NC (normal control) group and 119 people in SZ (schizophrenia) group, and accuracy, sensitivity, and specificity of classification results were evaluated.

A positive symptom classification model of the second classification model classified 57 people in HPSZ (high positive schizophrenia) group and 62 people in LPSZ (low positive schizophrenia) group, and accuracy, sensitivity, and specificity of classification results were evaluated.

A negative symptom classification model of the second classification model classified 55 people in HNSZ (high negative schizophrenia) group and 64 people in LNSZ (low negative schizophrenia) group, and accuracy, sensitivity, and specificity of classification results were evaluated.

A cognitive/disorganization symptom classification model of the second classification model classified 59 people in HCSZ (high cognitive/disorganization schizophrenia) group and 60 people in LCSZ (low cognitive/disorganization schizophrenia) group, and accuracy, sensitivity, and specificity of classification results were evaluated.

In this case, brain activity data on seven brain regions of frontal lobe, insula, temporal lobe, occipital lobe, tempo-occipital lobe, parietal lobe, and limbic lobe was used for learning and evaluation of each model. However, types of brain activity data used for learning of the first classification model and the second classification model are not limited thereto.

Furthermore, brain activity data corresponding to 11 frequency domains of delta waves from 1 to 4 Hz, (δ), theta waves (θ) from 4 to 8 Hz, alpha waves (α) from 8 to 12 Hz, alpha 1 waves from 8 to 10 Hz, alpha 2 waves from 10 to 12 Hz, beta waves (β) from 12 to 30 Hz waves, beta 1 waves from 12 to 18 Hz, beta 2 waves from 18 to 22 Hz, beta 3 waves from 22 to 30 Hz, beta 4 waves from 18 to 30 Hz, and gamma waves (γ) from 30 to 55 Hz were used for learning and evaluation of each model. However, types of brain activity data used for learning of the first classification model and the second classification model are not limited thereto.

Referring to FIG. 3C, the first classification model learned to classify SZ and NC is shown to have excellent classification performance, with accuracy of 80.66%, sensitivity of 78.83%, and specificity of 82.48% in classification. In particular, the positive symptom classification model of the second classification model is shown to have excellent classification performance for levels of symptoms, with accuracy of 88.10%, sensitivity of 88.40%, and specificity of 87.77%.

Referring to FIG. 3D together, the first classification model (SZ vs. NC) has an AUC value of 0.86, showing that it has excellent prediction performance for determining whether schizophrenia occurs. Furthermore, it is shown that in the second classification model, an AUC value of the positive symptom classification model (HPSZ vs. LPSZ) is 0.87, an AUC value of the negative symptom classification model (HNSZ vs. LNSZ) is 0.68, and an AUC value of the cognitive/disorganization symptom classification model (HCSZ vs. LCSZ) is 0.74. In other words, these results may mean that the second classification model has excellent classification performance of classifying subtypes according to levels of schizophrenia symptoms.

Accordingly, the first classification model can classify and provide with high reliability whether the individual's schizophrenia has occurred, and the second classification model can classify and provide the subtype of the individual's schizophrenia with high reliability.

Referring to FIGS. 3E and 3F, in each classification model, features of the brain activity data selected for classification of positive symptoms, negative symptoms, and cognitive/disorganization symptoms are listed.

First, referring to FIG. 3E, in the classification of normality and schizophrenia by the first classification model, it is shown that features of the frontal lobe were selected most frequently among the seven brain regions of frontal lobe, insula, temporal lobe, occipital lobe, tempo-occipital lobe, parietal lobe, and limbic lobe, followed by features of occipital lobe>limbic lobe>temporal lobe=parietal lobe. In the classification of HPSZ and LPSZ by the positive symptom classification model of the second classification model, features of the frontal lobe were selected most frequently, followed by features of tempo-occipital lobe>temporal lobe=limbic lobe=parietal lobe. In the classification of HNSZ and LNSZ by the negative symptom classification model of the second classification model, it is shown that the features of the frontal lobe, tempo-occipital lobe, and parietal lobe were selected most frequently. Furthermore, in the classification of HCSZ and LCSZ by the cognitive/disorganization model of the second classification model, the features of the parietal lobe were selected most frequently, followed by the features of the frontal lobe.

Referring to FIG. 3F, in the classification of normality and schizophrenia by the first classification model, it is shown that features of theta and beta 3 among the plurality of frequency domains, were selected most frequently, followed by features of delta>alpha>beta 2. In the classification of HPSZ and LPSZ by the positive symptom classification model of the second classification model, it is shown that features of an alpha frequency domain were selected most frequently, followed by the features of the delta. In the classification of HNSZ and LNSZ by the negative symptom classification model of the second classification model, it is shown that features of an alpha 2 frequency domain were selected most frequently. Furthermore, in the classification of HCSZ and LCSZ by the cognitive/disorganization model of the second classification model, it is shown that features of a beta 2 frequency domain were selected most frequently.

According to the above results, in classifying the levels of symptoms by each model, features of the five brain regions (ranked 1st to 5th) frequently selected, further, features of frequency bands of the six frequent bands (ranked 1st to 6th) selected can be selected for learning of the first classification model and the second classification model, but are not limited thereto.

The present invention can overcome limitations of a schizophrenia diagnosis system based on fMRI, which still has many limitations, such as providing of low-reliability information, high analysis costs, and spatial and temporal constraints.

Additionally, the present invention can allow medical staff to provide information about an individual, enabling continuous monitoring of the individual suspected of having schizophrenia.

Therefore, the present invention can contribute to early diagnosis and good treatment prognosis of schizophrenia by providing information on whether schizophrenia has occurred.

Although the exemplary embodiments of the present invention have been described in detail with reference to the accompanying drawings, the present invention is not limited thereto and may be embodied in many different forms without departing from the technical concept of the present invention. Therefore, the exemplary embodiments of the present invention are provided for illustrative purposes only but not intended to limit the technical concept of the present invention. The scope of the technical concept of the present invention is not limited thereto. Therefore, it should be understood that the above-described exemplary embodiments are illustrative in all aspects and do not limit the present invention. The protective scope of the present invention should be construed based on the following claims, and all the technical concepts in the equivalent scope thereof should be construed as falling within the scope of the present invention.

### EXPLANATION OF REFERENCE NUMERALS

100: Device for providing information about schizophrenia

110, 230: Storage unit

120, 210: Communication unit

130, 240: Processor

200: User mobile device

220: Display unit

300: Medical staff device

400: Device for measuring brain waves

410: Brain wave data

412: Brain wave data form which noise has been removed

520: Brain activity data

522: Feature data

530: First classification model

532: Classification result

540: Second classification model

540a: Positive symptom classification model

540b: Negative symptom classification model

540c: Cognitive/disorganization Symptom Classification Model

542a: Positive symptom classification result

542b: Negative symptom classification result

542c: Cognitive/disorganization symptom classification result

1000: System for providing information about schizophrenia

[National R&D project supporting this invention]

[Project ID number] 1711110648

[Project number] 2018R1A2A2A05018505

[Government department name] Ministry of Science and ICT

[Project management (professional) organization name] National Research Foundation of Korea

[Research business name] Individual Basic Research (Ministry of Science and ICT) (R&D)

[Research project name] Development of prediction and diagnostic tool for mental illness using EEG and HRV and machine learning

[Contribution rate] 95/100

[Host organization] Inje University

[Research period] Mar. 1, 2020 to Feb. 28, 2021

[National R&D project supporting this invention]

[Project ID number] 1345304620

[Project number] 2019R1A6A3A01096980

[Government department name] Ministry of Education

[Project management (professional) organization name] National Research Foundation of Korea

[Research project name] Establishment of scientific and engineering research base (R&D)

[Research project title] Development of diagnosis technology of subtype by symptom of schizophrenia patients

using machine learning techniques

[Contribution rate] 5/100

[Host organization] Korea advanced institute for science and technology (KAIST)

[Research period] Sep. 1, 2019 to Aug. 31, 2020

1. A method for providing information about schizophrenia, implemented by a processor, the method comprising:
receiving an individual's brain wave data;
generating brain activity data based on the brain wave data;
determining whether the individual's schizophrenia has occurred, using a first classification model configured to predict whether schizophrenia has occurred by taking the brain activity data as input; and
when schizophrenia is predicted, determining symptoms of the individual's schizohrenia using a second classification model configured to classify symptoms of schizophrenia by taking the brain activity data as input.

2. The method of claim 1, wherein the second classification model is at least one of a positive symptom classification model configured to classify levels of positive symptoms by taking the brain activity data as input, a negative symptom classification model configured to classify levels of negative symptoms by taking the brain activity data as input, and a cognitive/disorganization symptom classification model configured to classify levels of cognitive/disorganization symptoms by taking the brain activity data as input.

3. The method of claim 1, further comprising:
after the determining of the symptoms of schizophrenia,
determining a prognosis of the individual according to the symptoms of the individual's schizophrenia.

4. The method of claim 1, further comprising:
after the generating of the brain activity data,
extracting features of the brain activity data,
wherein the determining of whether schizophrenia has occurred, includes determining whether the individual' schizophrenia has occurred based on the features, using the first classification model.

5. The method of claim 4, wherein the brain activity data includes a plurality of pieces of brain activity data, and
wherein the extracting of the features includes,
determining functional connectivity between the plurality of pieces of brain activity data, and
determining the features between the pieces of brain activity data based on network structural features of the functional connectivity.

6. The method of claim 5, wherein the determining of the functional connectivity includes,
determining connectivity of a phase locking value (PLV) for each of the plurality of pieces of brain activity data, and
wherein the determining of the features between the pieces of brain activity data includes determining the features based on a clustering coefficient and a path length for the connectivity of the PLV for each brain activity data.

7. The method of claim 1, further comprising:
filtering the brain activity data based on a band pass filter, which is performed after the generating of the brain activity data.

8. The method of claim 1, wherein the brain wave data is defined as brain wave data obtained in a resting state.

9. The method of claim 1, wherein the generating of the brain activity data includes converting the brain wave data into the brain activity data, by using at least one of MNE (minimum-norm estimate), LORETA (low-resolution brain electromagnetic tomography), sLORETA (Standardized low-resolution brain electromagnetic tomography), eLORETA (Exact resolution brain electromagnetic tomography), and dSPM (Dynamic statistical parametric mapping).

10. The method of claim 1, wherein the brain activity data includes current source density (CSD) in at least one brain region among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal.

11. A device for providing information about schizophrenia, comprising:
a communication unit configured to receive an individual's brain wave data; and
a processor connected to communicate with the communication unit,
wherein the processor is configured to,
based on the brain wave data, generate brain activity data,
determine whether the individual's schizophrenia has occurred, using a first classification model configured to predict whether schizophrenia has occurred by taking the brain activity data as input, and
when schizophrenia is predicted, determine symptoms of the individual's schizohrenia using a second classification model configured to classify symptoms of schizophrenia by taking the brain activity data as input.

12. The device of claim 11, wherein the second classification model is at least one of a positive symptom classification model configured to classify levels of positive symptoms by taking the brain activity data as input, a negative symptom classification model configured to classify levels of negative symptoms by taking the brain activity data as input, and a cognitive/disorganization symptom classification model configured to classify levels of cognitive/disorganization symptoms by taking the brain activity data as input.

13. The device of claim 11, wherein the processor is further configured to determine a prognosis of the individual according to the symptoms of the individual's schizophrenia.

14. The device of claim 11, wherein the processor is further configured to,
extract features of the brain activity data, and
determine whether the individual' schizophrenia has occurred based on the features, using the first classification model.

15. The device of claim 14, wherein the brain activity data includes a plurality of pieces of brain activity data, and
wherein the processor is configured to,
determine functional connectivity between the plurality of brain activity data, and
determine the features between the pieces of brain activity data based on network structural features of the functional connectivity.

16. The device of claim 15, wherein the processor is configured to,
determine connectivity of a phase locking value (PLV) for each of the plurality of pieces of brain activity data, and
determine the features based on a clustering coefficient and a path length for the connectivity of the PLV for each brain activity data.

17. The device of claim 11, wherein the processor is further configured to filter the brain activity data based on a band pass filter.

18. The device of claim 11, wherein the brain wave data is defined as brain wave data obtained in a resting state.

19. The device of claim 11, wherein the processor is configured to convert the brain wave data into the brain activity data, by using at least one of MNE (minimum-norm estimate), LORETA (low-resolution brain electromagnetic tomography), sLORETA (Standardized low-resolution brain electromagnetic tomography), eLORETA (Exact resolution brain electromagnetic tomography), and dSPM (Dynamic statistical parametric mapping).

20. The device of claim 11, wherein the brain activity data includes current source density (CSD) in at least one brain region among banks of the superior temporal sulcus, caudal anterior cingulate, caudal middle frontal, cuneus, entorhinal, frontal pole, fusiform, inferior parietal, inferior temporal, insula, isthmus cingulate, lateral occipital, lateral orbito frontal, lingual, medial orbito frontal, middle temporal, para central, para hippocampal, pars opercularis, pars orbitalis, pars triangularis, pericalcarine, post central, posterior cingulate, precentral, precuneus, rostral anterior cingulate, rostral middle frontal, superior frontal, superior parietal, superior temporal, supramarginal, temporal pole, and transverse temporal.
